(19) United States
(12) Patent Application Publication
(10) Pub. No.: US 2006/0211665 A1
(43) Pub. Date: Sep. 21, 2006

(54) Reduction of postoperative pain medication

(76) Inventors: Chitranjan S. Ranawat, Alpine, NY (US); Amar S. Ranawat, New York, NY (US)

Correspondence Address: SCHWEITZER CORNMAN GROSS & BONDELL LLP, 292 MADISON AVENUE - 19th FLOOR, NEW YORK, NY, 10017, US

(21) Appl. No.: 11/083,183

(22) Filed: Mar. 17, 2005

(51) Int. Cl. A61K 31/573; A61K 31/485; A61K 31/24; A61K 31/137

(52) U.S. Cl. 514/171; 514/282; 514/534; 514/649

(57) ABSTRACT

A procedure for reducing the pain from orthopedic surgery, such as hip or knee replacement by injecting a mixture of components into the site of trauma (surgical incision or tissue twisting/stretching) before closing the wound. The mixture includes a local anesthetic agent, epinephrine, morphine and a corticosteroid.

### BACKGROUND OF THE INVENTION

Postoperative pain control is often the most challenging and troublesome aspect of total joint replacement surgery. Inadequate pain control can be a source of significant anxiety and concern on the part of patients and their families. Additionally, recovery from surgery in terms of function, rehabilitation and discharge may be adversely affected by inadequate pain control.

Current protocols for postoperative pain management incorporate various combinations of parenteral narcotics, regional anesthesia and nerve blocks. During the first 24-48 hours after surgery, the dosages of narcotics administered by these routes can be significant and are associated with frequent side effects including nausea, itching, vomiting, drowsiness, urinary retention and ileus. Regional anesthesia and nerve blocks may be associated with temporary or permanent neurologic dysfunction. More serious side effects which are seen with narcotic use include respiratory depression and death. Additionally, their effectiveness is quite often sub-optimal. Ineffectiveness of PCA has been reported to be as high as 50% and rebound pain is a common occurrence.

As a result, there has been recent interest in providing more local pain control using a variety of local anesthetic agents via varying intra-articular delivery systems, such as infusion pumps, absorbable release capsules and sutures. The present invention differs from these delivery systems in that it represents a never-before-described technique for the infiltration of local anesthetic agents into the critical soft tissues around the hip and knee joint, and not merely an intra-articular release of these agents.

### BRIEF DESCRIPTION OF THE INVENTION

The present invention serves to significantly reduce the need for pain medication after surgery, particularly in orthopedic surgery, such as knee, hip or other joint replacement, and thereby hasten the recovery of function. An advanced postoperative pain management protocol called “Reduced Tissue Trauma Surgery” RTTS has been developed.

The present invention represents a novel technique for the infiltration of a proprietary mixture of local anesthetic agents into the critical soft tissues around the hip and knee joint, and significantly reduces the need for pain medication after surgery (particularly orthopedic surgery such as knee or hip replacement).

### DETAILED DESCRIPTION

The agents for local infiltration used in the present invention have all been approved for local infiltration and are widely used in the medical field. Preferred concentrations are shown below with suitable ranges shown in parenthesis (dry weight). All dosages are well below documented toxic amounts for each individual agent. Although it has not been specifically studied to date, the use of these combined agents with this technique may have an additive effect.
(1) any long activity local anesthetic agent, such as bupivicaine (marcaine), lidocaine, or Novocain;
(2) epinephrine;
(3) morphine, opiates, duramorph, and other slow-release morphine derivatives;
(4) corticosteroids, such as Depo-Medrol, Betamethasone Diproprionate, prednisolone, Triamcinolone Acetonide, Triamcinolone Hexacetonite, lidocaine;
(5) any first or second-generation cephalosporin.
   A typical mixture for injection will contain (based on weight percent):
10 mg to 500 mg, preferably 80 to 120 mg local anesthetic agent;
100 mcg to 500 mcg, preferably 200 mcg to 300 mcg epinephrine;
2 mg to 20 mg, preferably 4 mg to 6 mg morphine;
20 mg to 200 mg, preferably 40 mg to 80 mg corticosteroids;
0 to 2 gm, preferably 500 mg to 1000 mg antibiotics;
   Specific formulations are as follows:

16/24cc of 0.5% Bupivicaine (Marcaine/Sensorcaine) (80/120 mg)—This is their most widely used long acting local anesthetic for postoperative pain control. The dosage as been determined after factoring in the dose of local anesthetic typically given for the spinal/epidural. This dose has been determined by the anesthesiology team to be will within the safe limit for the average adult. The dose is verified with the anesthesiologist on a case-by-case basis as an additional safety precaution. (40 to 120 mg).

0.3 cc of 1:1000 Epinephrine (300 mcg)—Epinephrine is routinely used in conjunction with bupivicaine for infiltration because of its ability to prolong its effectiveness by decreasing its absorption through its alpha-adrenergic actions. Additionally, there may be a secondary advantage via its vasoconstrictive effect in decreasing bleeding in the wound. The secondary effect is not being investigated in this study.

6 mg of Morphine—Increasingly, recent literature has proven that morphine has effectiveness for pain blockade via peripheral action. The central nervous system effects of morphine have been shown to be more effective in conjunction with its peripheral blockade. Additionally, it is well accepted that it has excellent analgesic effect when used in regional anesthesia. Addition of morphine to this mixture of agents is thought to further supplement analgesia via a completely different mechanism than the other agents used. (4 to 6 mg).

40 mg of methylprednisolone (depo-medrol)—This is a potent anti-inflammatory agent. It is well established that methylprednisolone modifies the inflammatory response to surgical trauma via numerous inflammatory mediators. Much of the postoperative pain is related to the body's response to surgery with resultant inflammation and edema. By decreasing this inflammation, edema, swelling and pain, narcotic requirements should be less and recovery should improve.

Cefuroxime (Zinacef) 750 mg prepared in 10 cc normal saline (0.9%)—This additive is not expected to improve pain after infiltration but is expected to decrease the chances of infection. This is done in conjunction with the standard peri-operative intravenous antibiotic prophylaxis and antibiotic irrigation solution. Cefuroxime is in a similar class of antibiotics used for intravenous antibiotic prophylaxis (usually Cefazolin—Ancef/Kefzol).

Normal Saline (0.9%)—for dilution and ease of infiltration.

The following mixture of agent is then combined under sterile procedures in a normal saline solution for a total volume of 60 cc. This mixture is injected by needle in a manner never-before-described in the medical literature into the soft tissues around the hip and knee. Whereas other FDA-approved, local delivery systems currently available focus on the intra-articular release of anesthetic agents, this system relies on the soft tissue penetration of these agents to gradually elute their effects. Moreover, by concentrating the agents in the soft tissues which elicit the greatest pain response the effect on recovery of function is enhanced.

Injection Technique:

After stable joint reconstruction with trial implants is achieved, the trials are removed and the wound is copiously irrigated. Removal of the implants allows for increased access to the peri-articular soft tissues, particularly the posterior capsule for the knee and the anterior capsule for the hip. Using proper injection technique (aspiration prior to injection) to avoid intravascular injection, the posterior capsule of the knee is insufflated with approximately 25% of the total solution using multiple passes from medial to lateral. The remaining 75% is injected in a step-wise fashion into the insertions and origins of the medial and lateral collateral ligaments, the synovium, and the cut edge of the quadriceps tendon. For the hip, 25% of the solution is injected into the anterior capsule while the remaining 75% is injected into the insertion of the gluteus medius, the posterior capsule and short external rotators, the gluteus medius, and the cut edges of the fascia lata.

The main benefit of the present injection-technique is overall improvement in postoperative pain control. If the need for parenteral narcotics can be reduced or eliminated, there will be less narcotic related side effects or adverse events. Additionally, the expenses associated with maintaining a PCA or epidural pump will be eliminated. Improved postoperative rehabilitation, improved functional recovery and shorter hospitalizations are also potential benefits of the present protocol.

The following examples will serve to illustrate the present invention. All parts and percentages in said examples and elsewhere in the specification are by weight unless otherwise indicated.

### EXAMPLES

From Oct. 1, 2003 through Jun. 20, 2004, 50 patients (50 hips) and 36 patients (52 knees) underwent total hip and total knee replacement with an advanced perioperative pain management protocol. (RTTS). All patients were given spinal anesthesia.

Intraoperatively, a 10-20 cm incision was made using sharp dissection to avoid stretching, tearing, and maceration of the skin and underlying facial planes. Prior to closure, a local proprietary mixture of Marcaine 80 mg, Depo-Medrol 40 mg, Morphine 4 mg, Epinephrine 300 mcg, Zinacef 750 mg and Clonidine 100 mg was injected into the periarticular ligamentous attachments, synovium, capsule, and orthrotomy sites. The sites of the surgical incision the tissues are twisted or stretched. Patients were followed with postoperative pain scales and monitored for narcotic requirements. Additionally, patient assessment questionnaires were used to document recovery of functional milestones, such as unassisted walking, stair-climbing, range-of-motion (ROM), and overall satisfaction.

Results

During the study period, narcotic pain requirements, manipulation rates, and the need for prolonged physical therapy were significantly reduced as compared to historical controls. Recovery of functional milestones and ROM was achieved at an earlier period in 90% of patients. Overall patient satisfaction was greatly improved.

By controlling acute pain in the critical early postoperative period (three days) following total hip and total knee replacement, the RTTS pain management protocol allowed for improved recovery of functional milestones and improved patient satisfaction. It appears that pain control plays a much larger role in functional recovery than incision length.

Various modifications can be made to the present invention without departing from the essence thereof, such as the use of other local anesthetic agents or applying this technique to other surgical procedures.

What is claimed is:

1. A method for reducing postoperative pain discomfort in orthopedic surgery which comprises after incision but before wound closure injecting a mixture of agents containing a local anesthetic into the site where a trauma has been created by the operation.

2. The method of claim 1 used in hip replacement.

3. The method of claim 2 used in knee surgery.

4. The method of claim 1 wherein the trauma site for injection is the site of the surgical incision.

5. The method of claim 1 wherein the trauma site is where tissues are stretched or twisted.

6. The method of claim 1 wherein said mixture of agents comprises a local anesthetic, morphine, epinephrine and methylprednisolone.

7. The method of claim 1 wherein said local anesthetic agent is bupivicaine (marcaine).

8. A method for reducing pain discomfort in orthopedic surgery which comprises making a 10-20 cm incision using a sharp dissection to avoid stretching, tearing and maceration of the skin, after incision but before wound closure injecting a mixture of marcaine, morphine, epinephrine, cefuroxine (zinacet), and clonidine into the sites where the tissues are twisted or stretched.

9. The method of claim 8 which serves to control acute pain in the early postoperative period (three days).

10. The method of claim 8 used to control pain discomfort in hip replacement and knee surgery.
